SEQUENCE LISTING (3 sequences)

SEQ ID NO: 1 - protein, 12 aa, Artificial
  Binding motif
GFMSWEDNACSE

SEQ ID NO: 2 - protein, 29 aa, Artificial
  Dual binding motif
GFMSWEDNACSEGSGSGSGFMSWEDNACS

SEQ ID NO: 3 - DNA, 25 bp, Artificial
  nucleic acid sequence of molecular beacon.
gcgactttgggtttgggtttctcgc

(19) United States
(12) Patent Application Publication
(10) Pub. No.: US 2004/0158051 A1
(43) Pub. Date: Aug. 12, 2004

(54) Mono and dual conjugation of nanostructures and methods of making and using thereof

(76) Inventors: Mihri Ozkan, San Diego, CA (US); Joong Hyun, Riverside, CA (US)

Correspondence Address: Suzannah K. Sundby, Smith, Gambrell & Russell, Suite 800, 1850 M Street, N.W., Washington, DC, 20036, US

(21) Appl. No.: 10/715,501

(22) Filed: Nov. 19, 2003

Related U.S. Application Data
(60) Provisional application No. 60/427,700, filed on Nov. 19, 2002.
(60) Provisional application No. 60/427,776, filed on Nov. 19, 2002.
(60) Provisional application No. 60/428,009, filed on Nov. 20, 2002.

(51) Int. Cl. C12Q 1/68; C07H 21/04

(52) U.S. Cl. 536/23.1; 435/6

(57) ABSTRACT

Disclosed herein are molecular beacons comprising a quantum dot attached to a first end of at least one nucleic acid molecule having a probe and forms a stem-loop structure in the absence of a target sequence hybridized thereto and a quencher attached to a second end of the nucleic acid molecule. Also disclosed are methods for making the molecular beacons, molecular beacon arrays, and kits comprising the molecular beacons.

### CROSS REFERENCE TO RELATED APPLICATIONS

This application claims the benefit of U.S. Provisional Patent Application No. 60/427,700, filed 19 Nov. 2002, 60/427,776, filed 19 Nov. 2002, and 60/428,009, filed 20 Nov. 2002, all of which list Mihri Ozkan and Joong Hyun Kim as the inventors, and are herein incorporated by reference in their entirety.

### BACKGROUND OF THE INVENTION

1. Field of the Invention

The present invention generally relates to mono and dual conjugated nanostructures and methods of making and using thereof.

2. Description of the Related Art

In geology minerals are deposited by precipitation from solution or by solidification from melts, in biology inorganics are grown through biomineralization within or on an organic matrix that exerts a strong influence on the shape of the inorganic structure (e.g., the spiral minarets of snails, eggshells, abalone shells, tooth enamel, bone, etc). See BIOMIMETIC MATERIALS CHEMISTRY (1996) ed. S. Mann (VCH, WEinheim); and Addadi, L. and S. Weiner Angewandte Chemie 31:153-169. The degree of control in biomineralization extends over a wide range of length scales, from the atomic to the macroscopic, e.g. from the type of ion packing to skeletal structures. Crystal growth in biology is not left to the thermodynamics of inorganic crystal chemistry alone; it is controlled and guided by organic molecules. This is necessary as the crystalline materials in biology serve many different purposes, for example to form a protective, hard layer with a shape sympathetic to the way that an organism as a whole grows. Organic layers achieve this influence by acting as a template imposing constraints on how inorganic ions stick to their surfaces. These constraints maybe based on charges, e.g. a positive calcium ion will prefer to stick to an acidic carboxylate group or through the arrangement of binding sites, e.g. epitaxial growth will start from the crystal plane matching the imposed lattice distances. See Bianconi, P. A. et al. (1991) Nature 349:315. Today's human ceramic engineering feats are stellar but many important aspects of natural mineralization methods are still elusive and traditional manufacturing methods are starting to reach their limits.

### SUMMARY OF THE INVENTION

The present invention generally relates to molecular beacons.

In some embodiments, the present invention provides a molecular beacon comprising a quantum dot attached to a first end of at least one nucleic acid molecule having a probe and forms a stem-loop structure in the absence of a target sequence hybridized thereto and a quencher attached to a second end of the nucleic acid molecule. In some embodiments, the quantum dot is a ZnS capped CdSe quantum dot. In some embodiments, the quencher is an organic quencher. In some preferred embodiments, the quencher is DABCYL. In some preferred embodiments, the quencher is a gold substrate.

In some embodiments, the present invention provides a molecular beacon comprising a quantum dot attached to a first end of at least one nucleic acid molecule having a probe and forms a stem-loop structure in the absence of a target sequence hybridized thereto and a quencher attached to a second end of the nucleic acid molecule, wherein the quantum dot is a ZnS capped CdSe quantum dot and the quencher is DABCYL.

In some embodiments, the present invention provides a molecular beacon comprising a quantum dot attached to a first end of at least one nucleic acid molecule having a probe and forms a stem-loop structure in the absence of a target sequence hybridized thereto and a quencher attached to a second end of the nucleic acid molecule, wherein the quantum dot is a ZnS capped CdSe quantum dot and the quencher is a gold substrate.

In some embodiments, quantum dot of the molecular beacon of the present invention has two or more nucleic acid molecules having a probe and forms a stem-loop structure in the absence of a target sequence hybridized thereto and a quencher attached to a second end of the nucleic acid molecule. In some embodiments, the nucleic acid molecules are attached to the quantum dot in a pattern. In some embodiments, the nucleic acid molecules may be the same or different. In some embodiments, the probes may be the same or different.

In some embodiments, the molecular beacon may comprise two or more quantum dots that may be the same or different. In some embodiments, at least one quantum dot is a ZnS capped CdSe quantum dot.

In some embodiments, the molecular beacon may comprise two or more quenchers that may be the same or different. In some embodiments, at least one quencher is DABCYL or a gold substrate.

In some embodiments, the quantum dot and the quencher of the molecular beacon are operably linked, wherein in the absence of the target sequence hybridized thereto, the quencher quenches the quantum dot signal.

In some embodiments, the present invention provides a molecular beacon array comprising a plurality of molecular beacons according to the present invention.

In some embodiments, the present invention provides a kit comprising a molecular beacon of the present invention and instructional material.

It is to be understood that both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention and together with the description serve to explain the principles of the invention.

### DESCRIPTION OF THE DRAWINGS

This invention is further understood by reference to the drawings wherein:

FIG. 1 summarizes the method of the present invention for the mono-conjugation of nanostructures.

FIG. 2A shows functionalization of nanoparticles.

FIG. 2B shows selective functionalization of nanoparticles (multiple functional groups).

FIG. 3A shows formation of multi-particle network.

FIG. 3B shows formation of multi-particle network.

FIG. 4 shows the electric field based functionalization of a protein layer with protein protrusions.

FIG. 5A shows an active microelectronic array with 100 test sites.

FIG. 5B shows an active microelectronic array with 400 (wafer) test sites.

FIG. 5C shows an active microelectronic array with 10,000 test sites.

FIG. 6 shows checker-boarding of fluorescent DNA molecules by alternating the DC electric field on a microelectronic array (bias changed every 6 seconds).

FIG. 7A shows an example of biomineralization on an organic template.

FIG. 7B shows how the shell of a mollusc (Nautilus repertus) might form.

FIG. 8 is a schematic illustration of an electrode geometry and fluidic cell with adjustable height transparent counter electrode and reference electrode positioned above the working electrode array.

FIG. 9 illustrates the design of ceramic with embedded photovoltaic cells using quantum dots and carbon nanotubes.

FIG. 10A shows a prior art MB structure.

FIG. 10B shows a hybrid inorganic and organic MB of the present invention which comprises a QD in place of an organic fluorophore.

FIG. 11A shows a MB array without target sequence.

FIG. 11B shows a MB array with target sequence.

FIG. 12 schematically shows how to make the MBs according to the present invention.

FIG. 13A shows a gel electrophoresis of hybrid-MBs. From the left to right: lane MB, 267 pmole of non-conjugated MB (control); lane QD, modified quantum dots; lane QDMB, quantum dot conjugated MBs (initial amount of MB; 8 nmole, initial amount of modified QDs; 11 nmole). Ethidium bromide staining for 10 minutes was used to visualize MBs. In lane QDMB, the position of the band which was higher than the band in lane MB confirms the covalent bond between the modified quantum dots and MBs. Notice that there were no other bands which indicated the uniformity of the final reaction product.

FIG. 13B shows a comparison of the absorption spectra of DABCYL with the emission spectra of the mercaptoacetic acid modified QDs and commonly used organic fluorophore, 6-Fam. The excitation wavelength for QD and 6-Fam were 350 nm and 495 nm, respectively. About 90% and 35% overlap was observed between the spectra's of QD and DABCYL and 6-Fam and DABCYL, respectively. During the quenching of fluorescent signal (the “off” state of MB) the amount of overlap is a significant factor when the quenching occurs by the FRET mechanism.

FIG. 14A shows the 3D molecular model of MB of the present invention. The separation distance between the center of QD and the N═N bond of DABCYL is about 3.3 nm.

FIG. 14B shows the 3D molecular model of MB of the present invention. The separation distance between the center of QD and the N═N bond of DABCYL is about 5 nm.

FIG. 15A shows a comparison of the fluorescence detected for QD modified MB and 6-Fam attached MB. Background signal of the buffer solution was recorded first and then MBs without their target sequences were recorded

FIG. 15B shows hybrid MB hybridization analysis with specific and non-specific target sequences.

### DETAILED DESCRIPTION OF THE INVENTION

The present invention provides methods for conjugating nanostructures or microstructures. As used herein, “affixed”, “attached”, “associated”, “conjugated”, “connected”, “immobilized”, and “linked” are used interchangeably and encompass direct as well as indirect connection, attachment, linkage, or conjugation unless the context clearly dictates otherwise.

When a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. When a value being discussed has inherent limits, for example if a component can be present at a concentration of from 0 to 100%, or if the pH of an aqueous solution can range from 1 to 14, those inherent limits are specifically disclosed. When a value is explicitly recited, it is to be understood that values, which are about the same quantity or amount as the recited value, are also within the scope of the invention. When a combination is disclosed, each subcombination of the elements of that combination is also specifically disclosed and is within the scope of the invention. Conversely, when different elements or groups of elements are disclosed, combinations thereof are also disclosed. When any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

As used herein, the term “nanoparticle”, “nanostructure”, “nanocrystal”, “quantum dot”, and “nanocomponent” are used interchangeably to refer to a particle, generally a semiconductive or metallic particle, having a diameter in the range of about 1 nm to about 1000 nm, preferably in the range of about 2 nm to about 50 nm, more preferably in the range of about 2 nm to about 20 nm (for example about 2, 3, 4, 5, 6, 7, 8, 9, 10, 11, 12, 13, 14, 15, 16, 17, 18, 19, or 20 nm). Nanostructures include molecular beacons (MBs), quantum dots (QDs), carbon nanotubes (CNTs), colloidal nanocrystals, colloidal polymers, colloidal metal or semiconductor particles, hollow or filled nanobars, magnetic, paramagnetic, conductive or insulating nanoparticles, synthetic particles, hydrogels (colloids or bars), and the like. A QD has size dependent properties, e.g. chemical, optical, and electrical properties, along three orthogonal dimensions. A QD can be differentiated from a quantum wire and a quantum well, which have size-dependent properties along at most one dimension and two dimensions, respectively. It will be appreciated by one of ordinary skill in the art that QDs can exist in a variety of shapes, including but not limited to spheroids, rods, disks, pyramids, cubes, and a plurality of other geometric and non-geometric shapes. While these shapes can affect the physical, optical, and electronic characteristics of QDs, the specific shape does not bear on the qualification of a particle as a QD. A QD typically comprises a “core” of one or more first materials and can optionally be surrounded by a “shell” of a second material. Although thiol stabilized ZnS capped CdSe QDs are exemplified herein, other suitable QDs such as CdSe, TiO₂, and the like may be used according to the present invention. In some preferred embodiments, the QDs of the present invention are ZnS capped CdSe QDs. N-type QDs can be made by successful electron transfer from sodium biphenyl to the LUQCO (Lowest Unoccupied Quantum-Confined Orbital) of the nanocrystals. See Shim, M. et al. (2000) Nature 407:981, which is herein incorporated by reference.

Suitable materials for the core and/or shell of QDs, include a first element selected from Groups 2 and 12 of the Periodic Table of the Elements and a second element selected from Group 16 (e.g., ZnS, ZnSe, ZnTe, CDs, CdSe, CdTe, HgS, HgSe, HgTe, MgS, MgSe, MgTe, CaS, CaSe, CaTe, SrS, SrSe, SrTe, BaS, BaSe, BaTe, and the like); materials comprised of a first element selected from Group 13 of the Periodic Table of the Elements and a second element selected from Group 15 (GaN, GaP, GaAs, GaSb, InN, InP, InAs, InSb, and the like); materials comprised of a Group 14 element (Ge, Si, and the like); materials such as PbS, PbSe and the like; and alloys and mixtures thereof. As used herein, all reference to the Periodic Table of the Elements and groups thereof is to the new IUPAC system for numbering element groups, as set forth in the Handbook of Chemistry and Physics, 81 st Edition (CRC Press, 2000), which is herein incorporated by reference.

The QDs of the present invention may be optionally surrounded by a an organic capping agent. The organic capping agent may be any number of materials, but has an affinity for the QD surface. In general, the capping agent can be an isolated organic molecule, a polymer (or a monomer for a polymerization reaction), an inorganic complex, or an extended crystalline structure. The coat can be used to convey solubility, e.g. the ability to disperse a coated QD homogeneously into a chosen solvent, functionality, binding properties, or the like. In addition, the coat can be used to tailor the optical properties of the QD.

The present invention provides at least one nanostructure conjugated to a substrate or another nanostructure and methods for mono and dual conjugation. As used herein, the terms “substrate” and “support” are used interchangeably and refer to a material upon which a given structure such as a MB or QD of the present invention may be attached or immobilized.

The substrate can comprise a wide range of material such as biological material, nonbiological material, organic material, inorganic material, and the like, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonate, and the like, or combinations thereof. The substrates may be planar crystalline substrates such as silica based substrates including glass, quartz, or the like, or crystalline substrates such as those used in the semiconductor and microprocessor industries which include silicon, gallium, arsenide, and the like. Silica aerogels can also be used as substrates, and can be prepared by methods known in the art. Aerogel substrates may be used as free-standing substrates or as a surface coating for another substrate material.

The substrate may be in any form and typically is a plate, slide, bead, pellet, disk, particle, strand, precipitate, membrane, optionally porous gel, sheets, tube, sphere, container, capillary, pad, slice, film, chip, multiwell plate or dish, optical fiber, or the like. The surface of the substrate can be etched using well known techniques to provide for desired surface features such as trenches, v-grooves, mesa structures, and the like. The surfaces on the substrate can comprise the same material as the substrate or may be made from a material different from the substrate which may be affixed thereto by chemical or physical methods known in the art. The materials of the substrate surfaces may comprise a variety of materials such as polymers, plastics, resins, polysaccharides, silica or silica-based materials, carbon, metals, inorganic glasses, membranes, or any of the above-listed substrate materials. One skilled in the art may readily select the substrate and/or substrate surface materials in order to obtain a desired characteristic using methods and know-how in the art.

The present invention also provides methods for synthetic biomineralization, i.e. making a mineral phase within or on top of an organic substrate or matrix, and products made therefrom.

Generally, as disclosed herein, a dense array of individually controllable electrodes is used to position and/or grow organic substrates or matrices, initiate and accelerate inorganic crystal growth, guide nanostructures or nanocomponents to specific positions and regulate the self-assembly of nanostructures. Whereas prior art electronic arrays have relatively large metal electrodes, greater than about 10 micrometers diameter Au or Pt electrodes, the arrays of the present invention are preferably scaled down to where the size of the electrodes becomes about the same or substantially the same size of some of the nanostructures or nanocomponents to be manipulated, less than about 1 μm. In some embodiments, the electrodes are made of carbon (C-MEMS) such as those in the prior art.

The dense array may be used to functionalize nanostructures such as nanocrystalline particles, nanocomponents, or templates with high precision to assist their assembly into higher order three-dimensional structures. For example, the dense array may be used for mono- or dual- conjugation of nano-ceramic bars, colloids, nucleic acid molecules, and the like, which offers highly reliable and controlled specific attachment during the assembly process.

As used herein, “polynucleotide”, “oligonucleotide”, “nucleic acid”, and “nucleic acid molecule” are used interchangeably herein to refer to a polymeric form of nucleotides of any length, and may comprise ribonucleotides, deoxyribonucleotides, analogs thereof, or mixtures thereof. These terms refer only to the primary structure of the molecule. Thus, these terms include, for example, 3′-deoxy-2′,5′-DNA, oligodeoxyribonucleotide N3′P5′ phosphoramidates, 2′-O-alkyl-substituted RNA, triple-, double-, and single-stranded deoxyribonucleic acid (DNA), as well as triple-, double-, and single-stranded ribonucleic acid (RNA). The terms also include hybrids of nucleic acid molecules including hybrids between DNA and RNA or between PNAs and DNA or RNA, and also include known types of modifications, for example, labels, alkylation, “caps”, substitution of one or more of the nucleotides with an analog, intenucleotide modifications such as those with uncharged linkages, e.g methyl phosphonates, phosphotriesters, phosphoramidates, carbamates, and the like, with negatively charged linkages, e.g. phosphorothioates, phosphorodithioates, and the like, and with positively charged linkages, e.g. aminoalkylphosphoramidates, aminoalkylphosphotriesters, those containing pendant moieties, such as proteins including enzymes, toxins, antibodies, signal peptides, poly-L-lysine, and the like, those with intercalators, those containing chelates, those containing alkylators, and those with modified linkages. The terms also include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing nonnucleotidic backbones, for example, polyamide, e.g. peptide nucleic acids (PNAs) and polymorpholino (commercially available from the Anti-Virals, Inc., Corvallis,Oreg. as Neugene®) polymers, and other synthetic sequence-specific nucleic acid polymers providing that the polymers contain nucleotides in a configuration which allows for base pairing and base stacking, such as is found in DNA and RNA.

“Complementary” or “substantially complementary” refers to the ability to hybridize or base pair between nucleotides or nucleic acids, such as, for instance, between the two strands of a double stranded DNA molecule or between a polynucleotide primer and a primer binding site on a single stranded nucleic acid to be sequenced or amplified. Complementary nucleotides are, generally, A and T (or A and U), or C and G. Two single-stranded RNA or DNA molecules are said to be complementary when the nucleotides of one strand, optimally aligned and compared and with appropriate nucleotide insertions or deletions, pair with at least about 80% or more of the nucleotides of the other strand, preferably at least about 90% or more, more preferably at least about 95% or more, and even more preferably about 98% or more.

Substantial complementarity exists when an RNA or DNA strand will hybridize under selective hybridization conditions to its complement. Typically, selective hybridization will occur when there is at least about 65% or more complementary over a stretch of at least 14 to 25 nucleotides, preferably at least about 75% or more, more preferably at least about 90% or more complementary. See Kanehisa (1984) Nucleic Acids Res. 12:203, which is herein incorporated by reference.

Although methods known in the art may be used to construct and template various nanostrutures, preferred approaches include (1) active electronic arrays, (2) nano-particle tagging, (3) nucleation/electrocrystallization and scaling of electrokinetic phenomena, (4) genetically engineered protein templates, and (5) C-MEMS, and are described herein.

1. Nano-Particle Tagging

Controlled electric fields can be used not only to guide but also to orient nanostructures into well-organized patterns. The AC and DC fields allow the basic nanostructure core to be selectively derivatized with biological and/or chemical ligands, i.e. nanostructures are functionalized with specific binding or functional groups arranged in a desired pattern such as tetrahedral, hexagonal or other coordinate positions around the core nanostructure.

Prior art nanofabrication methods do not allow most nanostructures to be modified in a controlled or precise manner. While prior art methods allow a higher-order nanostructure to be formed, there is little or no control as to how the nanostructures or nanocomponents are arranged around the core nanostructure. Furthermore, prior art methods do not allow binding or functional groups of two or more types to be patterned around the core nanostructure. Prior art methods cannot be used for specific placement and attachment of binding or functional groups. In addition, prior art methods cannot avoid the multiple placement of functional groups on one given nanostructure which results in multiple nanostructures being bound to the given nanostructure.

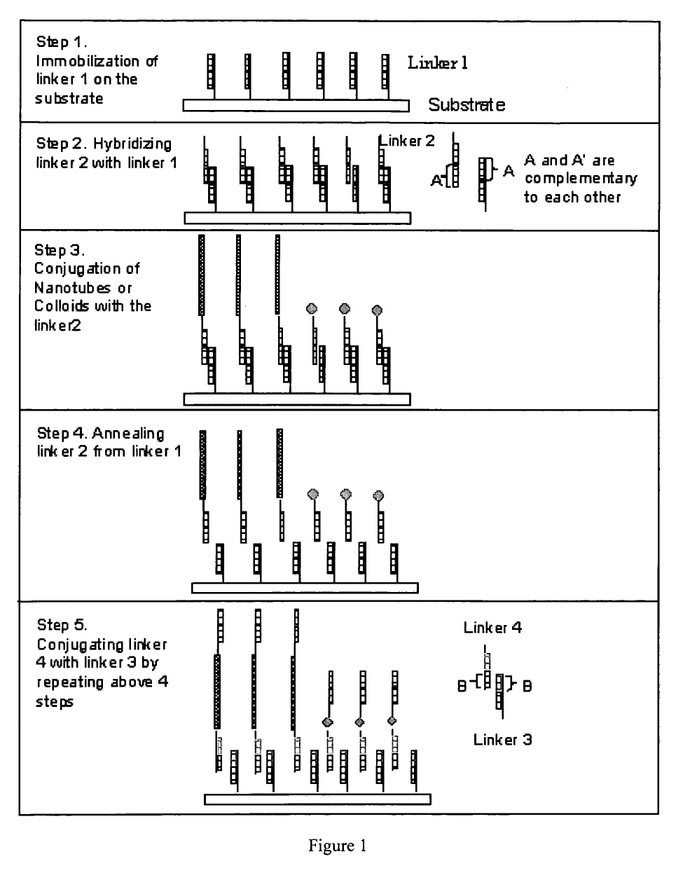

The present invention provides mono- or dual- conjugation of nanostructures, which offers highly reliable and controlled specific attachment during the assembly process. FIG. 1 schematically illustrates a method of the present invention for the monoconjugation of nanostructures and nanocomponents. As shown in FIG. 1, a monolayer of a first linker comprising a first nucleic acid molecule (A) is immobilized or attached to a substrate such as gold or silicon using methods known in the art. The first linker is immobilized or attached to the substrate such that the first nucleic acid molecule may hybridize with nucleic acid molecules having complementary sequences. Then a second linker comprising a second nucleic acid molecule (A′) which has a sequence that is complementary to the first nucleic acid molecule is hybridized to the first nucleic acid molecule using methods known in the art. The conditions for hybridization are preferably moderate hybridization conditions, more preferably, stringent hybridization conditions as commonly understood and practiced in the art.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions of about 6×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from about 5 minutes to about 24 hours, with 1 or more washing steps, and wash incubation times are about 1 to about 15 minutes. SSC is 0.15 M NaCl and 15 mM citrate buffer. It is understood that equivalents of SSC using other buffer systems can be employed.

As used herein, “nucleic acid probe” and “probe” are used interchangeably and refer to a structure comprising a polynucleotide, as defined above, which contains a nucleic acid sequence that can bind to a corresponding target. The polynucleotide regions of probes may be composed of DNA, and/or RNA, and/or synthetic nucleotide analogs.

As used herein, “preferential binding” or “preferential hybridization” refers to the increased propensity of one nucleic acid molecule to bind to a complementary nucleic acid molecule in a sample as compared to noncomplementary nucleic acid molecules in the sample or as compared to the propensity of the one nucleic acid molecule to form an internal secondary structure such as a hairpin or stem-loop structure under at least one set of hybridization conditions.

The free end of the second linker comprises a functional group such as a thiol group or an amino group. Then a nanostructure or nanocomponent having a functional group such as a carboxyl group is linked or attached to the free end of the second linker via covalent bonding between the functional groups. Then the bonds between the first nucleic acid molecule and the second nucleic acid molecule are annealed using methods known in the art which include heating the nucleic acid molecules to a temperature about their given melting points, raising the pH to more than about 11, or applying a negative voltage. See Ozkan, M. et al. (2003) Special issue on the Biomolecular Interface, Langmuir 19(5):1532-1538; and Ozkan, M. et al. (2001) IEEE Journal of EMB Magazine 20(6):144-151, which are herein incorporated by reference. Alternatively, the first linker and the second linker may be separated using a restriction enzymes as known in the art. Where use of restriction enzymes are desired, the nucleic acid molecules are designed such that they comprises the desired restriction enzyme sites according to methods known in the art. Use of restriction enzyme sites enable a third nucleic acid molecule to be ligated thereto using ligases and methods known in the art.

To ensure mono-conjugation of nanoparticles, the packing density of the first nucleic acid molecule immobilized or attached the substrate is important. Generally, the maximum packing density of nucleic acid molecules on a surface is less than about 1 molecule per 1 nm² (about 1 molecule per 100 Å²) on a highly smooth substrate. Therefore, the packing density of the first nucleic acid molecule is adjusted by using longer nucleotide sequences, e.g. about 20-mer oligonucleotides to about 40-mer oligonucleotides, thereby causing strong Columbic repulsion between adjacent nucleic acid molecules on the substrate. In the embodiments of the present invention, the size of the substrates is in the nanometer range, thereby ensuring the mono-conjugation of particles.

By controlling the packing density of a nucleic acid molecule on a smooth substrate, the number of nucleic acid molecules that may be used to attach further nanostructures may be adjusted. For small particles about 5 nm one nucleic acid molecule per particle can be attached via a covalent bond using methods known in the art, the nucleic acid molecule attachment to the substrate can be broken using using methods known in the art, thereby resulting in a single nucleic acid molecule attached to a nanoparticle. The method of the present invention eliminates further attachment of many other nanoparticles while forming a network of multiple nanoparticles, thereby ensuring single attachment of a second nanoparticle to a single nucleic acid molecule attached nanoparticle.

Similarly, where binding of two different types of nanoparticles are required, the single nucleic acid molecule attached nanoparticle may be dual-functionalized. A second nucleic acid molecule may be attached to the opposite side of the single nucleic acid molecule attached nanoparticle which is attached on a substrate. At the end, two different types of nanoparticles with complementary nucleic acid sequences functionalized around them can be attached to the opposite ends of this central dual-functionalized nanoparticle. Biomimetic of organic-inorganic materials can be fabricated from the “bottom-up” using the methods of the present invention. Electronic, opto-electronic, photonic devices or other useful structures may be made according to the methods of the present invention.

In some embodiments, a nanostructure having a plurality of layers may be made by a similar method and using nucleic acid molecules having two different sequences attached to two different positions on the nanostructures. A nucleic acid molecule having a sequence complementary to the first sequence on a first nanostructure is attached to a substrate and then the first nanostructure having the first sequence is then hybridized thereto. Then a second nanostructure having a sequence complementary to the second sequence is hybridized to the second sequence to attached the second nanostructure to the first nanostructure. This process may be repeated to link multiple nanostructures or to provide multiple layers of nanostructures. The nanostructures may be the same or different.

An active electrode array may be used to assist the assembly of the mono- and dual- conjugated nanostructures. Either AC or DC electrokinetics may be employed in the placement of the tagged nanostructures. The current and voltage may be controlled such that the placement of the nanostructures are modulated via local mass transport to given sites on the forming structure. The linkers on the nanostructures may be removed or disintegrated by exposure to high temperatures.

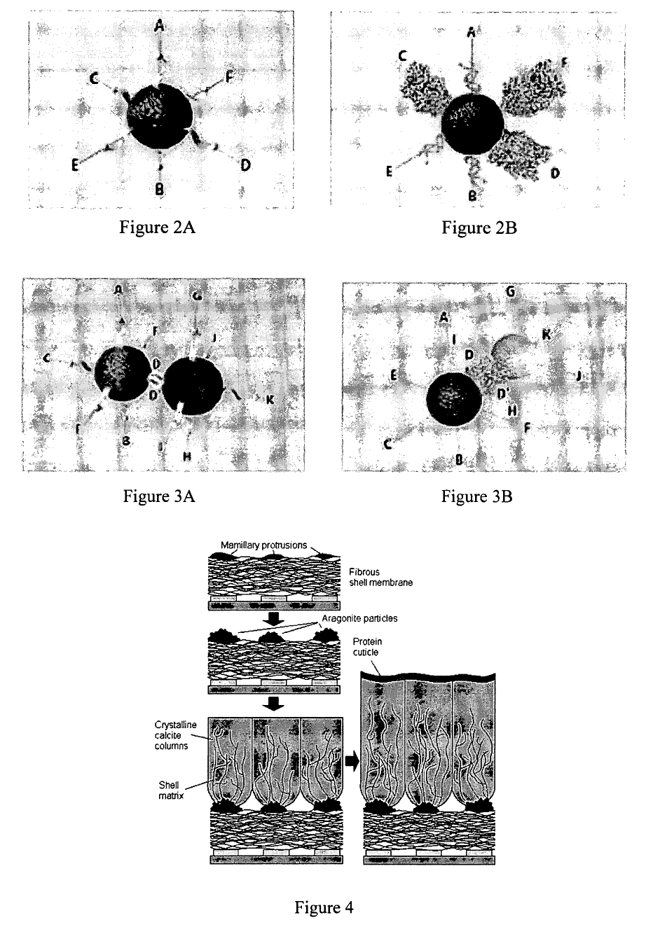

FIGS. 2A and 2B show how core nanostructures may be selectively modified. In this case, the nanostructures may be derivatized at given positions around the core with specific nucleic acid sequences, proteins, other biological ligands or chemical entities. Such modifications will allow a given nanostructure to be joined with another nanostructure in a more precise manner, i.e. leaving other selected positions on the nanostructure available for binding different nanostructures as shown in FIGS. 3A and 3B.

FIG. 4 illustrates the electrical field tagging principle in which the formation of columnar egg shell material was induced. In eggshells, which are produced much faster than seashells, calcite crystals are stacked in columns perpendicular to the egg's surface. The crystals are nucleated around nodules of protein on the surface of a fibrous organic membrane, and the calcite grows in columns, interwoven with strands of a protein matrix, up from the nucleation sites. See Hincke, M. T. et al. (2000) Chapter 36 in EGG NUTRITION AND BIOTECHNOLOGY Eds. J S Sim, S Nakai and W Guenter CAB International Wallingford, UK, pp. 447-461, which is herein incorporated by reference.

According to the present invention, an electronic array may be used, nodules of protruding protein are deposited above a selected set of electrodes, and columnar growth is initiated above each of these protruding protein micro-locations. By electrophoretically accumulating protein nodules over a set of otherwise homogenous fibrous shell membrane coated electrodes, one can create protein nodules on top of which the mineral deposition first starts and columnar crystal growth can be induced. Besides the deposition of the tagging protein on the fibrous shell layer, the electrical field further shapes the columns of aragonite growing on top of the protein protrusion by supplying calcium ions principally from the top direction. The latter can be arranged by the correct placement of counter and working electrodes in the array.

2. Active Electronic Arrays

Active microelectronic arrays that use AC and DC fields to create geometries for transporting and positioning both charged and uncharged particles, including DNA, biological cells, antibodies, enzymes, polymer particles, LEDs, and the like may be used according to the present invention See Heller M. J., et al. (2002) in INTEGRATED MICROFABRICATED DEVICES, Marcel Dekker, Eds. Heller and Guttman, Chap. 10, pp.223-270; and Edman C. F., et al. (2000) IEEE Photonics Technology Letters 12(9): 1198-1200, which are herein incorporated by reference.

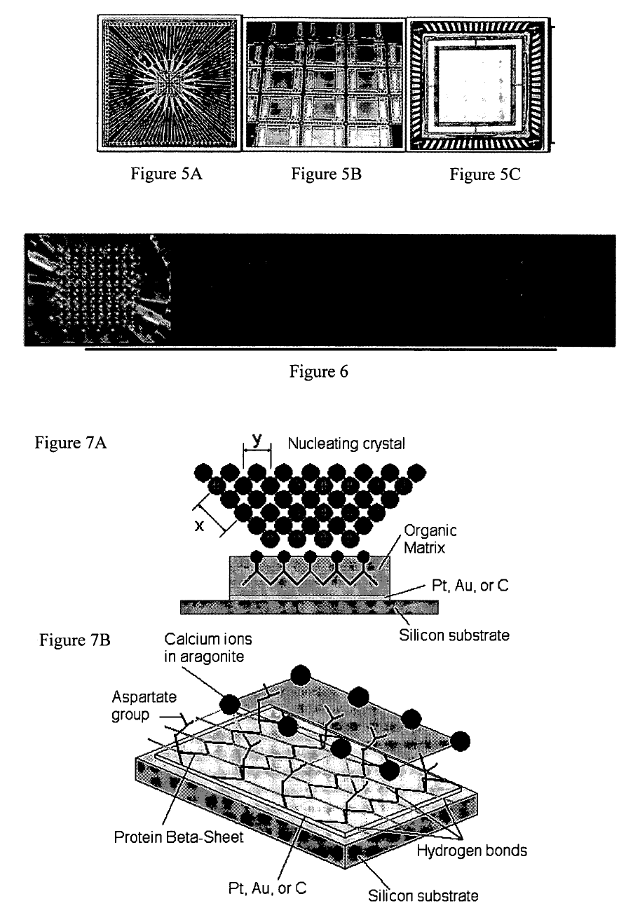

In some embodiments, the active microelectronic arrays are fabricated with 25, 100, 400, and 10,000 test-sites or microlocations. An example of a 100 test-site chip, has been commercialized (Nanogen, San Diego, Calif.) and has 80 μm diameter test-site/microlocations with underlying platinum microelectrodes, and twenty auxiliary outer microelectrodes. The outer group of microelectrodes provides encompassing electric fields for concentrating charged particles from the bulk sample solution to the activated test sites/microlocations on the surface. Each microelectrode has an individual wire interconnect through which current and voltage are applied and regulated. The 100 test-site array chip is preferably about 7 mm square in size, with the active test-site array area being about 2 mm square in size. See FIG. 5A. The active microchips are generally fabricated on silicon wafers. The base structure or substrate is silicon with an insulating layer of silicon dioxide. The microelectrode structures are platinum and the connecting wires are gold. Silicon dioxide or silicon nitride is used to cover and insulate the conducting wires, but not the surface of the platinum microelectrode structures.

The active array surface is often covered with several microns of hydrogel (usually agarose or polyacrylamide), which forms a permeation layer. The permeation layer may be impregnated with a linker such as streptavidin, a chemical agent, or the like, that allows for the subsequent attachment of a probe, label, and the like, to the specific test site/microlocation. The fabrication process is scalable and arrays with smaller features and higher density of test sites/microlocations may be used. In FIG. 5B a 400 test-site device with 50 micron microlocations is demonstrated and FIG. 5C shows a 10,000 test-site device with 30 μm diameter electrodes. The 400 and 10,000 test site arrays represent a more sophisticated device that has on-chip CMOS control elements to regulate currents and voltages to each of the microlocations.

These microelectronic arrays may be used to conduct highly parallel and multiplex nucleic acid hybridization assays including genotyping, gene expression analysis, and immunoassays. See Heller M. J., et al. (2002) in INTEGRATED MICROFABRICATED DEVICES, Marcel Dekker, Eds. Heller and Guttman, Chap. 10, pp.223-270, which is herein incorporated by reference. As applied herein, the microelectronic arrays may be used to manipulate nanostructures and nanocomponents. For example, FIG. 6 shows the controlled parallel movement (checker-boarding) of negatively charged fluorescent nucleic acid molecules between alternating positively and negatively biased microlocations. The sample solution comprised a 50 nM concentration of a 20-mer oligonucleotide sequence labeled in the 5′ terminal position with Bodipy Texas Red fluorophore, the microelectrodes were biased positive and negative in a checkerboard fashion, and the field was reversed every six seconds. At about 3 V DC the fluorescent nucleic acid molecules (about 7 nm in length) were transported back and forth a distance of about 200 μm during the 6 second switching time period.

Microelectronic arrays have also been used to direct the binding of derivatized nanoparticles. For example, polystyrene nanospheres derivatized with specific oligonucleotides are transported and bound to selected microlocations derivatized with the specific complementary oligonucleotide sequences. Thus, microelectronic arrays are not just limited to selective transport and binding of small molecules such as nucleic acid molecules or proteins, but also for selective transport and addressing of larger nanoparticles, microspheres, cells and even 20 μm light emitting diodestructures. See Edman C. F. et al. (2000) IEEE Photonics Technology Letters 12(9):1198-1200, which is herein incorporated by reference. Thus, microelectronic arrays may be used as “hostboards” or “motherboards” for the conjugation of nanostructures according to the present invention. In the DC electric field mode the electrophoretic transport of charged molecules and nanostructures is carried out between the positive and negative biased microlocations. The rate of transport is related to the strength of the electric field and the charge/mass ratio of the molecule or structure. When the microelectronic arrays are used to generate high frequency AC fields they can carry out the process of dielectrophoresis (DEP). At high AC fields, uncharged nanostructures, cells, and other micron-scale structures may be oriented and selectively positioned based on their intrinsic dielectric properties (between or on electrodes). See Washizu, M. et al. (1994) IEEE Trans. Ind. Appl. 30:835-843; Smith, P. et al. (2000) Applied Physics Letters 77:1399-1401; Talary, M. S. et al. (1996) J. Phys. D: Appl. Phys. 29:2198-2203; Tsukahara, S. et al. (2001) Chemistry Letters 3:250-251; Schnelle, T. et al. (1996) Naturwissenschaften 83:172-176; and Pethig, R. (1996) Critical Reviews in Biotechnology 16(4):331-348, which are herein incorporated by reference.

FIG. 7 illustrates a method of using an active electronic array for synthetic biomineralization. Organic layers, which control crystallization, are deposited on a conductive electrode such as Pt, Au or C and the same conductor electrode is used to localize and accelerate the nucleation of a microcrystal on this templating organic film. See FIG. 7A. The increase in crystal growth is a result of the increase in mass transport through electrophoretic ion transport to the organic templating layer. By arraying the underlying conductor electrodes in specific geometries varying crystallite assemblies may be fashioned.

FIG. 7B illustrates how the shell of a mollusc (Nautilus repertus) might form. See Philip Ball (1999) MADE TO MEASURE: NEW MATERIALS FOR THE 21^{ST} CENTURY, Princeton University Press, which is herein incorporated by reference. Repeating aspartate groups, part of a protein β-sheet, are arranged at the right distance to epitaxially grow the aragonite material. Also, in the nacre in abalone shells, in which aragonite is deposited on a pleated β-sheet of a silk-like protein, the repeat unit of the negative groups almost matches in size the repeat distances between calcium ions in the aragonite crystallites. The interface between the mineral plates and the organic matrix in abalone shells is relatively weak but it is this weak interface that prevents a crack from propagating through the material (this is similar to bonding strong SiC slabs together with graphite sheets to create a though, fracture-resistant composite). Red abalone shells are nearly as fracture-resistant as synthetic ceramics such as zirconia, carbon boride, silicon carbide, and silicon nitride. See M. Sarikaya (1999) PNAS USA 96(25); T. Graham & M. Sarikaya (2000) Materials Sci. & Eng. C, 11:145-153, which are herein incorporated by reference.

The present invention provides a method for synthetic biomineralization. For example, according to the present invention one may selectively deposit structured organic templates that will alternate with inorganic nanostructures such as aragonite plates. Depending on the application of the resulting nanostructure the linker may be removed or remain as a binding layer. The organic guiding structures, when sacrificial, act just like a photoresist in traditional top-down manufacturing methods, with a major difference that the organic structures of the present invention are on the nanoscale and are localized and specific. The organic templating layers may be made to be conductive, have a sensing function, or both.

3A. Electrocrystallization and Nucleation

In electrocrystallization controlling current or voltage at an electrode/electrolyte interface produces a new solid phase. Because of the increased mass transport electrocrystallization methods provide high quality crystals in shorter times than diffusion based methods. Important factors that influence the deposition process include polarization effects, the energy and geometry of solvated ions, and the formation of a thin electrical double layer at the growth front or interface. See ELECTRODE PROCESSES, DISCUSSIONS OF THE FARADAY SOCIETY No.1:1947 Butterworths (1961); Schaefer, R. A. et al. (1952) 39:487; and Lowenheim, F. A. (1973) MODERN ELECTROPLATING 3^{rd} ed., John Wiley & Sons, which are herein incorporated by reference. The condition of the surface to be coated is a basic determining factor in the kinetics of the overall electrocrystallization process and the resulting morphology as well. The presence of other inorganic ions and organic additives in the double layer or adsorbed on to the surface can modulate the crystallization process dramatically. Several electrorecrystallization mechanisms are known and all of them involve the same fundamental steps. See Lowenheim, F. A. MODERN ELECTROPLATING 3^{rd} ed., John Wiley & Sons (1973); and Bunshah, R. F., HANDBOOK OF DEPOSITION TECHNOLOGIES Noyes Publications (1994), which are herein incorporated by reference.

First, ions from a solution are transferred or deposited as adions (adsorbed ions) to a surface site. Second, adions diffuse across the surface until they encounter a crystallizing edge or step where further adsorption takes place. Third, deposition of ions results in the depletion in the solution adjacent to the substrate surface. The depleted ions must be replenished if the electrocrystallization process is to continue. This can be accomplished in several ways, including ionic migration, convection or diffusion See Vetter, K. J. (1967) ELECTROCHEMICAL KINETICS, Academic Press, which is herein incorporated by reference. A variety of materials including metals, oxides, insulating organic films and several organic superconductors have been grown using the method of electrocrystallization. See Kassegne, S. et al. (2003) Journal of Sensors and Actuators B: Chemical B 94(1):81, which is herein incorporated by reference. An active microelectronic array described herein may be used to study the deposition processes of organic and inorganic materials and combinations thereof.

The initial stages in the electrodeposition of materials onto different substrates occur by the birth and three-dimensional growth of nuclei. During a short time interval after a potentiostatic pulse, applied to generate the nuclei, they grow free of interaction with neighbors. The current maxima in transients provide information required to evaluate the density of active sites on the surface and the rate of nucleation per active site. Different growth rates in perpendicular and parallel directions affect the formation and shape of the nuclei.

3B. Scaling of Electrokinetic Phenomena

The active electronic array may be optimized for the nanoscale using an ultra precision e-beam lithography. Optimization of array design will be measured by quantification of electrophoretic and dielectrophoretic transport and nucleation and crystallization of nanoparticles. The electrophoretic quantification measurements will be carried out optically using fluorescent markers attached to transport species. An important feature for the electronic array will be the implementation of multiplexed potentiostatic and galvanostatic operation with each electrode element part of a three-electrode system.

The two important design parameters for array optimization for electrophoretic transport are electrochemical control and electrode geometry design. It has been demonstrated that the geometry of the electrode elements plays an important role in electric field distribution and strength at array electrode locations and for a 2 dimensional electrode system, the field drops sharply above the plane of the electrodes. To investigate this further an electrode array configuration with a counter electrode (or arrays of counter electrodes) that can be set at various heights above the working electrode plane as shown schematically in FIG. 8 is used. The counter electrode is made from a transparent conductor so quantification and characterization of transport and accumulation can continue to be made optically.

Finite element based simulation has also been used successfully to get a detailed picture and understanding of the electric field distribution in dielectrophoretic (DEP). The effect of electrode size on E-field intensity was simulated. As the size of the electrode is reduced to the nanometer scale, i.e. 500 nm, a preliminary analysis with a fine mesh showed that the electric field intensity at the edges increase significantly. As these edges are areas of numerical difficulty, the finite element mesh used should be as refined as possible to capture this significant peak at the edges. The modeling of edge effect has been reported in the literature. See Ferrigno, R. et al. (1997) Electrochimica Acta 42(12): 1895-1903, which is herein incorporated by reference. In the case of dielectrophoresis, electrode scaling effects become important in the dielectric force upon small particle transport rates. The dielectric force on a particle is proportional to the particle radius cubed (r³). The dielectric force scales linearly with the effective polarizability of the particle with respect to its suspending medium. As particles become smaller their dielectrophoretic transport rate decreases. For particles with radii less than 100 nm, the gradient of the square of the electric field (quantifying the field non-uniformity) should be greater than 10¹⁷ V² m⁻³ in order to overcome the Brownian force. In large electrode systems, this would cause local heating leading to detrimental hydrodynamic effects. However, down scaling the electrode radius is beneficial for dielectrophoresis. For example, reducing the radius of curvature 100-fold produces a 1000 fold increase in the gradient of the square of the electric field. Lower potentials can thus be used to produce the same dielectrophoretic force on a particle in the same relative position. The volume of liquid energized by the electrode is large compared to the electrode cross sectional area, so heat produced by the electrical current is efficiently dissipated. These edge effects, therefore, assume additional importance in the case of electrodes of very small diameter, i.e. in the range of 10s and 100s of nanometers.

4. Genetically Engineered Protein Templates

Another approach that may be used to fabricate hybrid inorganic/organic nanomaterials is by employing bacteriophages to display peptides in their surface that recognize the target inorganic molecule. For example, this approach has been demonstrated in the preparation of peptides that bind semiconductor particles. See Seeman, Nadrian C., et al (2002) PNAS USA 99(suppl. 2):6451-6455; Lee, Seung-Wuk, et al. (2002) Science 296:892-895; Whaley, Sandra R., et al. (2000) Nature 405:665-668; and Service, Robert F. (2001) Science 294:2462-2463, which are herein incorporated by reference.

First, peptidic phases that can recognize a target inorganic material such as calcite, aragonite, hydroxyapatite, strontium sulfate, and the like are prepared using a phage display of random libraries of peptides to identify peptides capable of binding to a target material, such as a ceramic surface, with high affinity. The identified peptides should be capable of binding with different degrees of affinity inorganic molecules of the same chemical composition but of different crystal geometries (polymorfs). For example, the peptides chosen should be able to selectively distinguish between calcite and aragonite, both CaCO₃, and bind preferentially one over the other. Once the peptides that bind better to the target ceramic material are identified, simple structures are assembled by using these peptides as linkers or binders between the inorganic phases.

Two different strategies for the preparation and identification of peptides/small proteins that are capable of selectively recognizing and binding of a target inorganic molecule may be used. The first one is based on phage display techniques and the second one on biomimetic principles.

Phages are small-sized viruses that infect bacterial cells, and replicate in their bacterial hosts. These phages are used in a number of biotechnology applications as vectors to replicate a foreign desired DNA in bacterial hosts such as E. coli. As the phage vector replicates in its E. coli host, then, the foreign desired DNA “insert” carried by the phage replicates too. If the phage vector has been designed to be an expression vector, it has the property to express the foreign DNA as a peptide/protein. Thus, the DNA insert carried by the phage is recognized by the E. coli host's machinery and transcription and translation of the DNA results in the synthesis of a foreign peptide whose amino acid sequence is determined by the nucleotide sequence of the insert. Moreover, phage display vectors have an additional unique feature where the foreign DNA is genetically fused to the gene corresponding to one of the phage's coat proteins. This results in the expression of a hybrid fusion protein that displays the foreign peptide or protein domain on the outer surface of the bacterium. Phage display libraries are mixtures of phage clones, each carrying a different foreign DNA insert, and therefore, displaying a different peptide on its surface. See Smith, G. P. and Petrenko, V. A. (1997) Chem. Rev. 97:391-410, which is herein incorporated by reference. The process by which the peptides are screened for their binding ability toward a target molecule is known as biopanning and consists of four steps: (1) binding of phages to the target ceramic molecule, (2) washing to remove unbound phage, (3) dissociation to recover the substrate-specific phage, and (4) amplification of the ceramic-specific phage by infection of E. coli. The amplified phage is then isolated and reexposed to a new surface of a given substrate. The latter step results in the enrichment of the peptide that binds selectively and with the highest affinity to the target substrate. The procedure is repeated several times, typically about 3 to about 5 times. The final step involves the sequencing of the DNA of the phages that were selected based on this biopanning approach to determine the sequence of the selected binding peptide. Phage display is used for selection of peptides in a variety of applications such as drug discovery, bioanalysis, and the like, and kits for phage display and bio-panning are available from commercial sources such as New England Biolabs and Calbiochem. Libraries of 1.9×10⁹ random peptide sequences can be purchased and may be used to select for peptides that bind specifically to calcite, aragonite, hydroxyapatite, and strontium sulfate. Once the peptides have been identified and sequenced, they may be prepared in large amounts by employing genetic means or solid phase peptide synthesis. The binding of the peptide to a substrate may be characterized in terms of affinity, reversibility, and cross-selectivity to other materials that are similar in composition and in geometric shape.

The second strategy toward selection of such peptides is based on biomimetic principles and involves the rational design of the peptides by identifying natural proteins or binding motifs for a given substrate. Very recently, the role of certain proteins in the biomineralization and the mechanism of eggshell formation has been reported. See Lakshminarayanan, Rajamani, et al. (2002) PNAS 99:5155-5159, which is herein incorporated by reference. Eggshell biomineralization occurs by nucleation and deposition of layers of calcite crystals (CaCO₃) by proteins. It is known that active sites in the protein recognize the Ca²⁺ ions, inducing nucleation of a specific polymorf of CaCO₃. This controls the final morphology of the eggshell, i.e. calcite vs aragonite. The protein ansocalcin has recently been isolated and sequenced from goose eggshell. It is a small protein (15 kDa) rich in both, acidic amino acids (such as glutamic and aspartic acid residues) and basic amino acids (such as histidine, lysine, and arginine). It is believed that the acidic residues, which are arranged in six different pairs in ansocalcin, are potential Ca²⁺ binding motifs. Calcite crystals can grow in the presence of ansocalcin in a concentration-dependent manner.

The amino acid sequence of ansocalcin was studied and searched for proteins with sequence homology. Two proteins, factor X binding protein (X-bp) and tetranectin, were found. The X-bp protein is responsible for phospolipid membrane binding in the presence of Ca²⁺ ions. Like ansocalcin, X-bp also binds to calcium through acidic residues in the protein See Mizuno, Hiroshi, et al. (2001) PNAS 98:7230-7234, which is herein incorporated by reference. Tetranectin is a plasminogen-binding protein highly expressed during mineralization See Ibaraki, K., et al. (1995) Mamm. Genome 6:693-696, which is herein incorporated by reference. By using bioinformatics tools and matching databases of existing protein crystal structures, a 3D model for ansocalcin was prepared.

The X-ray crystal structure of tetranectin and the model structure of ansocalcin were overlaid to identify structure homology and amino acid Ca²⁺ binding motifs and it was found that the two proteins share significant structure homology, especially with regard to the Ca²⁺ binding sites. On the basis of this structural similarity a sequence of amino acids that will bind to Ca²⁺ and induce biomineralization may be constructed. Thus, the present invention provides peptides that can bind to a given substrate and induce the formation of nanocrystals by designing peptides comprising the moieties that induce binding.

For Ca²⁺ binding the peptide should provide anchoring for the CaCO₃ and guide nucleation, and later, formation of the calcite crystals. Different peptides will result depending on the chosen number of Ca²⁺ binding motifs and on the type and number of amino acids in the spacer. An example of an amino acid sequence that may be incorporated into “binding peptides” is as follows:

| | |
|---|---|
| GFMSWEDNACSE. | (SEQ ID NO: 1) |

The binding peptides may be prepared in E. coli by expression of a plasmid containing the nucleotide sequence encoding for the peptide. Conventional molecular biology techniques may be used for the construction of the plasmid and for the expression, isolation, and purification of the peptides.

As with the peptides selected by using phage display, the binding of the peptide to calcite will be characterized in terms of affinity, reversibility, and cross-selectivity to other materials that are similar in composition and in geometric shape. In this case, the binding ability may also be contrasted with that of the full ansocalcin protein that will be expressed in a similar manner as the peptides.

Dual binding peptides may be used to bring together inorganic phases in two different planes in a site-directed oriented fashion. This site-directed protein-mediated nucleation is different from work reported in the literature where protein-mediated nucleation is followed by aggregation leading to the formation of crystals. An example of a dual binding peptide has the following amino acid sequence:

| | |
|---|---|
| GFMSWEDNACSEGSGSGSGFMSWEDNACS. | (SEQ ID NO: 2) |

The Ca²⁺ binding domains are underlined and ununderlined is the spacer peptide. The two Ca²⁺ binding motifs are separated by a spacer composed of a series of glycines and serines (neutral amino acids) so that they do not interfere with each others binding ability. Therefore, the present invention provides dual binding peptides that have domains that specifically bind a given substrate.

Additionally, a binding peptide that has more that one 3D conformation may be used to for site directed orientation and placement of nanostructures. For example, a peptide having a first conformation in a certain environment, such as an acidic environment may be used to place a given nanostructure at point A. Then the environment may be changed, basic environment such that it induces the second conformation in order to place a given nanostructure at point B. The binding peptides of the present invention may be prepared and isolated according to methods known in the art.

5. C-MEMS

Gold and platinum electrode arrays may be used as nodes to generate DC and AC fields in a layer of solution and serve both as the actuators for the nano-manipulator platform and as anchor points for organic and ceramic nanoparticles. Gold and platinum electrodes may be fabricated with known IC sputtering techniques. The carbon electrodes may be fabricated directly from patterned photoresist using methods known in the art. Preferably, MEMS carbon electrodes are made by a pyrolysis process known in the art. See Kim, J., et al. (1998) J. Electrochem. Soc. 145(7):2314-2319; and Ranganathan, S., et al. (2000) J. Electrochem. Soc.147:277-282, which are herein incorporated by reference.

The nanostructures of the present invention may be used in optical devices and components. Crystals of interest for the manufacture of electroceramic components are anisotropic, however the polycrystalline forms in use today are isotropic before poling. Electric fields can be used to orient nanoparticles or rods during the nanoassembly process, so that the subsequent body will be anisotropic and photonic band gap devices may be constructed. Examples include a reflecting dielectric polarizer; a two-dimensional crystal that reflects all in-plane light within some specified frequency band, so it can be used as a band-stop filter. A defect mode in a photonic crystal would serve as an effective resonant cavity, since it would only trap light in a very narrow frequency band and would hardly suffer any losses. Waveguides can also be assembled; light that propagates in the waveguide with a frequency within the band gap of the crystal is confined to, and can be directed along a nanoassembled structure. A 2D organic matrix may be assembled on a conductive electrode, e.g. Pt or Au, which can localize and control the electrocrystallization process, e.g. of GaP, to form the 2D ceramic structure. By changing the shape of the templating organic matrix, the geometry of the 2D photonic band gap structure may be modified.

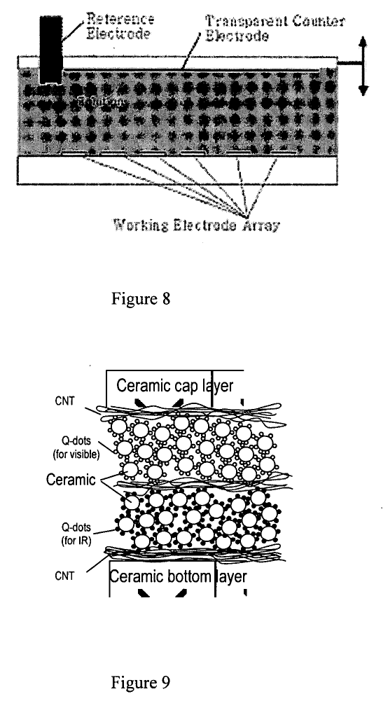

The nanostructures of the present invention may be used in artificial camouflage skin. In addition to energy generation and storage, one can use the properties of embedded QDs to change the color and reflectivity of nanostructured films akin to what happens in natural camouflage. When a bias voltage is applied across different layers of the QDs through carbon nanotube contacts as shown in FIG. 9, the electric-field can change the absorption spectrum of the QDs (creating a red-shift through the quantum-confined Stark effect). As a result, the “color” of the film can change with the applied voltage just like some insects or lizards change their skin color.

Hybrid Molecular Beacons

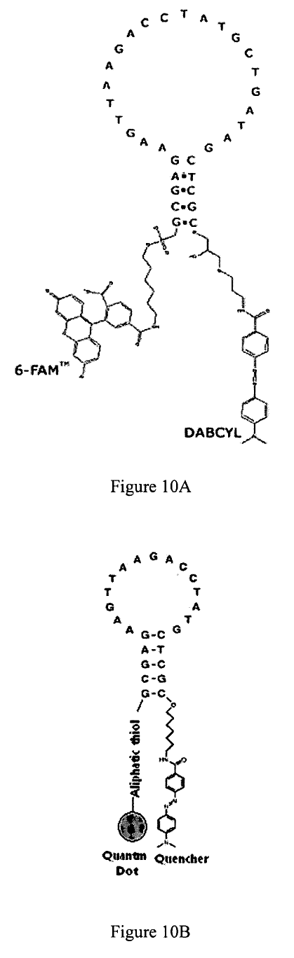

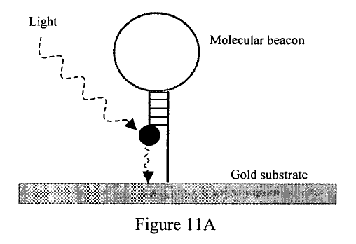

Use of fluorescent probes imparts many developments in molecular biology and medicine. See Pavski, V., and Le, X. C. (2003) Curr. Opin. Biotech. 14:65-73, which is herein incorporated by reference. Molecular beacons (MBs) are one of the unique deoxyribonucleicacid (DNA) and ribonucleicacid (RNA) probes that are at the “off” state when there is no complementary target sequence present and at the “on” state, when there is binding of the sequence that is under search. See Tyagi, S. and Kramer, F. R. (1996) Nat. Biotechnol. 14:303-308; Tyagi, S., et al. (1998) Nat. Biotechnol. 16:49-53; Kostrikis, L. G., et al. (1998) Science 279:1228-1229; Sokol, D. L., et al. (1998) PNAS 96:11538-11543; and Knemeyer, J. P., et al. (2000) Anal.Chem. 72:3717-3724, which are herein incorporated by reference. An illustration of a prior art MB is shown in FIG. 10A with 6-FAM™ (Midland Certified Reagent Company Midland, Tex.) as fluorescent reporter dye or donor and DABCYL as quencher or acceptor.

The present invention provides hybrid inorganic-organic MBs using QDs as fluorophores. See FIG. 10B. This technique overcomes the fast photobleaching of commercial MBs exhibit upon hybridization with target. Gel electrophoresis, fluorescent measurements and 3D molecular modelling results show the stability and the uniform structure of the MBs of the present invention. Given the specificity of MBs and the stability and multicolor properties of QDs, the MBs of the present invention may be employed in numerous molecular biology and molecular genetics applications such as assays and diagnostics known in the art.

The present invention provides a hybrid MB with inorganic fluorophore and organic quencher that exhibits improved stability against photobleaching. To this end, inorganic colloidal QDs after surface modification are attached to the 5′ end of MBs using methods known in the art. See Chan, W. C. and Shuming, N. (1998) Science. 281:2016; Alivisatos, A. P. (1996) Science 271:933; Niemeyer, C. M. (2001) Angew Chem Int Ed Engl. 40:4128-4158; Bruchez, M. Jr., et al. (1998) Science 281:2013-2015; Dabbousi, B. O., et al. (1997) J Phys Chem B. 101:9463-9475; Dahan, M., et al. (2001) Opt Lett. 26:825-827; Willard, D. M., et al. (1991) Nano Letters 1:467- 474; Wang, S., et al. (2002) Nano Letters 2:817-822; Akerman, M. E., et al. (2002) PNASUSA 99:12617-12627; and Mitchell, G. P., et al. (1999) J. Am. Chem. Soc 121:8122-8123, which are herein incorporated by reference. The quenching mechanism (fluorescence resonance energy transfer (FRET)) for this hybrid arrangement of QD with organic quencher is discussed herein and supported with 3D molecular modeling using methods known in the art. See Pathak, S., et al. (2001) J. Am. Chem. Soc. 123:4103-4104; Gerion, D., et al. (2001) J. Phys. Chem. B 105:8861-8871; Dubertret B., et al. (2002) Science 298:1759-1761; Mattoussi, H., et al. (2000) J. Am. Chem. Soc 122:12142-12150; Jaiswal, J. K., et al. (2003) Nature Biotech. 21:47-51; and Kagan C. R., et al. (1996) Phys. Rev. B 54:8633, which are herein incorporated by reference.

MBs have been frequently used in numerous applications including real-time monitoring of polymerase chain reactions, studying protein-DNA interactions, monitoring target RNAs in vivo for drug discovery and single-nucleotide-polymorphism (SNP) detection. See Vogelstein, B. and Kinzler, K. W. (1999) PNAS 96:9236-9241; Fang. X., et al. (2000) Anal.Chem. 72:3280-3285, Tsuji, A., et al. (2000) Bioohys. J. 78:3260-3274; and Ferentz, A. E. (2002) Pharmacogenomics 3:453-467; which are herein incorporated by reference. The structure of MBs provides high probe specificity, which is important when single base discrimination is investigated. However for different applications, MBs can be customized for optimal performance. In SNP studies high probe specificity is sufficient, but when studying transient RNA expression in vivo and in real time, fast hybridization kinetics are a more important quality. Some of these structural requirements can be an artifact of the choice of fluorophore and quencher. In commercial beacons, these components are both organic based materials. Use of organic fluorophores can limit the ability to observe target DNA or RNA in vivo as they have a limited lifetime. Organic fluorophores are also constrained by narrow absorbance spectra and broad emission spectra. Multicolor detection by MBs with organic fluorophores is very challenging as their broad emission spectra may overlap. Likewise, light sources of different wavelengths are required for excitation of these probes. For in vivo studies, this could be extremely damaging as over heating of live cells can occur with sequential illumination at different wavelengths.

In the present invention, semiconducting inorganic QDs are used to replace organic fluorophores. Recently developed mono-dispersed QDs offer substantial advantages over organic dyes. See Chan, W. C. and Shuming, N. (1998) Science 281:2016; Alivisatos, A. P. (1996) Science 271:933; Niemeyer, C. M. (2001) Angew Chem Int Ed Engl. 40:4128-4158; Bruchez, M. Jr., et al. (1998) Science 281:2013-2015; Dabbousi, B. O., et al. (1997) J Phys Chem B. 101:9463-9475; Dahan, M., et al. (2001) Opt Lett. 26:825-827; Willard, D. M., et al. (2001) Nano Letters 1:467- 474; Wang, S., et al. (2002) Nano Letters 2:817-822; Akerman, M. E., et al. (2002) PNASUSA 99:12617-12627; Mitchell, G. P., et al. (1999) J. Am. Chem. Soc 121:8122-8123; Pathak, S., et al. (2001) J. Am. Chem. Soc 123:4103-4104; Gerion, D., et al. (2001) J. Phys. Chem. B 105:8861-8871; Dubertret B., et al. (2002) Science 298:1759-1761; Mattoussi, H., et al. (2000) J. Am. Chem. Soc 122:12142-12150; Jaiswal, J. K., et al. (2003) Nature Biotech. 21:47-51; and Kagan C. R., et al. (1996) Phys. Rev. B 54:8633, which are herein incorporated by reference. QD color can be tuned by changing the particle size. QDs have narrow, symmetrical emission peaks allowing simultaneous multiplexed DNA/RNA detection. Most importantly, QDs are highly stable against photobleaching (about 20 times) permitting long term imaging. See Chan, W. C. and Shuming, N. (1998) Science. 281:2016, which is herein incorporated by reference.

The methods and materials disclosed above may be used to make a hybrid molecular beacon (MB) comprising a fluorophore, such as a QD, and an organic quencher or a substrate that quenches the fluorophore, such as a gold substrate. In preferred embodiments the QD is a ZnS capped CdSe QDs. As used herein, a “quencher” can be any material that can quench at least one fluorescence emission from an excited fluorophore being used in the assay; that is, any material that can cause the excited fluorophore to emit detectably less energy under the assay conditions. The quencher of the MBs of the present invention are organic quenchers such as DABCYL, BHQ-1, BHQ-2, BHQ-3, QSY 7, and the like. In preferred embodiments, the quencher is DABCYL. A number of suitable quenchers are known in the art and are commercially available.

QDs of the present invention may be made from any material and by any technique that produces QDs having emission characteristics useful in the methods, articles and compositions taught herein. The QDs have absorption and emission spectra that depend on their size, size distribution and composition. Suitable methods of production are disclosed in U.S. Pat. Nos. 6,048,616; 5,990,479; 5,690,807; 5,505,928; 5,262,357; 6,207,229; PCT Publication No. WO 99/26299; Murray et al. (1993) J. Am. Chem. Soc. 115:8706-8715; Guzelian et al. (1996) J. Phys. Chem. 100:7212-7219; Ridley et al. (1999) Science 286:746-749; Peng et al. (2001) 123:183-184; and Qu et al. (2001) Nano Lett. 1:333-337, which are herein incorporated by reference.

The surface of the QDs of the present invention may be modified to enhance emission efficiency by adding an overcoating layer to form a “shell” around the “core” QD using methods known in the art. See Dabbousi et al. (1997) J. Phys. Chem. B 101:9463; Kuno et al. (1977) J. Phys. Chem. 106:9869; Hines et al. (1996) J. Phys. Chem. 100:468-471; PCT Publ. No. WO 99/26299; U.S. Pat. No. 6,207,229; Danek et al. (1996) Chem. Mat. 8(1):173-180; Peng et al. (1997) J. Am. Chem. Soc. 119:7019-7029; Bruchez et al. (1998) Science 281:2013-2016 and U.S. Pat. No. 5,990,479which are herein incorporated by reference. Water-dispersible QDs can be prepared by methods known in the art. See PCT Publ. No. WO 00/17655; and U.S. Pat. No. 6,251,303, which are herein incorporated by reference. The surface layer of the QDs may be modified by displacement to render the QDs reactive for a particular coupling reaction using methods known in the art. See U.S. Pat. No. 5,990,479; Bruchez et al. (1998) Science 281:2013-2016, Chan et al. (1998) Science 281:2016-2018, which are herein incorporated by reference. The QD may be conjugated to other moieties directly or indirectly through a linker.

Examples of suitable spacers or linkers are polyethyleneglycols, dicarboxylic acids, polyamines and alkylenes. The spacers or linkers are optionally substituted with functional groups, for example hydrophilic groups such as amines, carboxylic acids and alcohols or lower alkoxy group such as methoxy and ethoxy groups. An example of a linker is a a nucleic acid molecule with a free thiol group at the end of 3′ or alkanethiol such as 6-mercapto-1-hexanol with longer or shorter thiol. Additionally, the spacers will have an active site on or near a distal end. The active sites are optionally protected initially by protecting groups. Among a wide variety of protecting groups which are useful are FMOC, BOC, t-butyl esters, t-butyl ethers, and the like. Various exemplary protecting groups are described in, for example, Atherton et al., Solid Phase Peptide Synthesis, IRL Press (1989), which is herein incorporated by reference.

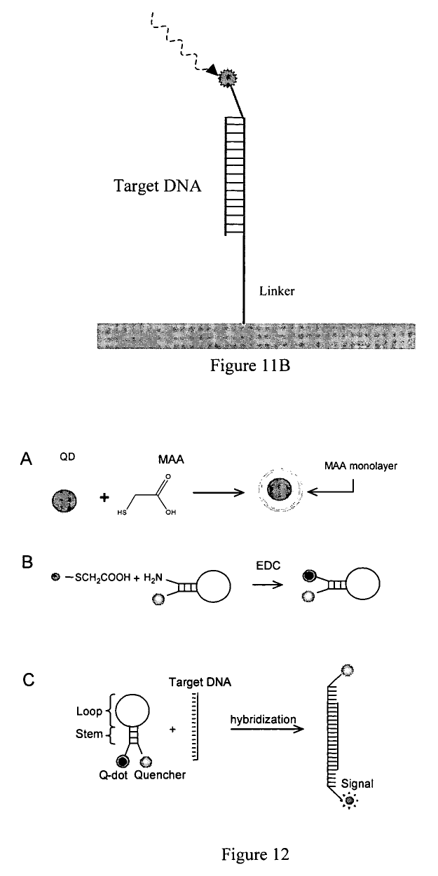

FIG. 10B is an illustration of a MB comprising a QD (ZnS capped CdSe) as the fluorophore and DABYCL as the organic quencher. FIG. 11A is an illustration of a MB comprising a QD as the fluorophore and a gold substrate which quenches the fluorophore where the fluorophore is in close proximity to the gold substrate. In both examples, the fluorophore is linked directly or indirectly at or in close proximity to a first end of a nucleic acid molecule which comprises a probe. The quencher is linked directly or indirectly at or in close proximity to a second end of the nucleic acid molecule or on a surface of a substrate with which the nucleic acid molecule is associated. The quencher and the fluorophore in this arrangement are of a type and are located such that the fluorescence emission from the fluorophore is quenched when the nucleic acid molecule forms a stem-loop structure, and the fluorescence emission from the fluorophore is not quenched when the probe is hybridized with a complementary sequence, e.g. target sequence. See FIG. 11B. Upon hybridization of the target sequence to the probe, a difference in the fluorescence of the fluorophore is detectable by at least one of the methods known in the art.

The target sequence may be single-stranded, double-stranded, or higher order, and can be linear or circular. Exemplary single-stranded target sequences include mRNA, rRNA, tRNA, hnRNA, ssRNA, ssDNA, and the like. Exemplary double-stranded target sequences include genomic DNA, mitochondrial DNA, chloroplast DNA, dsRNA, dsDNA, plasmids, phage, viroids, and the like. The target sequence may be synthetically prepared or obtained from a biological sample.

The nucleic acid molecule of the MB comprises a probe of interest. The nucleic acid molecule comprising the probe is optically silent but fluoresces upon hybridization with a complementary sequence, such as a target sequence. When the nucleic acid molecule comprising the probe is not hybridized with a complementary sequence, it forms a stem-loop structure where the loop portion is typically about 15 to about 30 nucleotides in length, but can be longer, and the stem portion is typically about 4 to about 7 nucleotides in length, but can also be longer. A fluorophore can be attached to one end of the nucleic acid molecule and a quencher can be attached to the other end. In the absence of a complement to the loop structure, the stem keeps the fluorophore and the quencher in close proximity to each other, thereby causing the fluorescence to be quenched by energy transfer. When a complementary sequence to the loop is present, the loop sequence will form a hybrid with the target sequence, thereby creating a linearized structure, which is longer and more stable than the stem-loop structure, thereby causing the fluorophore and the quencher to move away from each other which results in the restoration of fluorescence.

The probe may comprise all or part of the sequence of the nucleic acid molecule of the MB that forms the stem part of the stem-loop structure. Alternatively, the probe may only make up the loop part of the stem-loop structure. The only requirement for the probe hybridizes preferentially to the target sequence rather than forming the stem-loop structure. The length of the probe is not critical, but typically is from about 5 to about 100 nucleotides in length and is chosen to provide suitably selective binding to the desired target. The stem length, loop length, linker types and lengths, probe sequence, concentrations of samples and reagents, hybridization conditions, and incubation times may be optimized by one skilled in the art.

In preparing the hybrid MBs, first ZnS capped CdSe QDs of about 3.7 nm in diameter and about 490 nm at emission wavelength were treated to functionalize their surfaces by using mercaptoacetic acid as disclosed herein thereby giving them a negative surface charge. Surface modification of QDs by mercaptoacetic acid or other thiol compounds is widely used because of well worked out procedures. See Chan, W. C. and Shuming, N. (1998) Science 281:2016; Willard, D. M., et al. (2001) Nano Letters 1:467-474; Akerman, M. E., et al. (2002) PNAS USA 99:12617-12627; Mitchell, G. P., et al. (1999) J. Am. Chem. Soc. 121:8122-8123, which are herein incorporated by reference. Other techniques such as silanization, incapsulation with phospholipid micelles or use of proteins have been reported that improve the stability of QDs in aqueous solution. See Gerion, D., et al. (2001) J. Phys. Chem. B 105:8861-8871; Dubertret B., et al. (2002) Science 298:1759-1761; and Mattoussi, H., et al. (2000) J. Am. Chem. Soc 122:12142-12150, which is herein incorporated by reference. However, these methods increase the final size of nanocrystals by adding extra layers. This limits the efficiency of FRET which is the main mechanism of quenching the MB fluorophore and is inversely proportional with the 6^{th} power of the distance between donors (fluorophore) and acceptors (quenchers). See Van Der Meer, B. W., et al. (1994) RESONANCE ENERGY TRANSFER: THEORY DATA, VCH, New York, which is herein incorporated by reference.

Thus, the QDs of the present invention were functionalized according to the following: 0.5 ml of ZnS capped CdSe QDs (Dia. about 3.7 nm) purchased from Evident Technologies (Troy, N.Y.) in toluene solution were reacted with about 1.0 M mercaptoacetic acid (MAA) for at least 2 hours. During the reaction, QDs were precipitated by the exchange of TOPO with MAA. The MAA capped QDs were purified by centrifugation and washing with equal volume of chloroform 5 times. Later, chloroform was removed by evaporation at room temperature for a minimum of 2 hours. Next, surface modified QDs were resuspended in phosphate buffered saline (PBS) pH of 7.4. Through 1-ethyl-3-(3-dimethyl-aminopropyl) carbodiimide HCl (EDC) coupling, 10 μl of surface modified QDs were conjugated to about 1 μM of the 5′ amine terminated DNA sequence which is

| | |
|---|---|
| 5′ (NH₂C₆H₁₂)-GCGACTTTGGGTTTGGGTTTCTCGC | (SEQ ID |
| | NO: 3) |

and has a [4-(4′-dimethylaminophenylazo)benzoic acid] (DABCYL) at the 3′ end (Midland Certified Reagent Company Midland, Tex.) in 980 μl PBS, pH 8.2 for 24 hours at room temperature. For this reaction, EDC agent (10 fold mole more than MB) was used. After conjugation, purification was carried out by using Ultrafree®-0.5 Centrifugal Filter Units (Millipore).

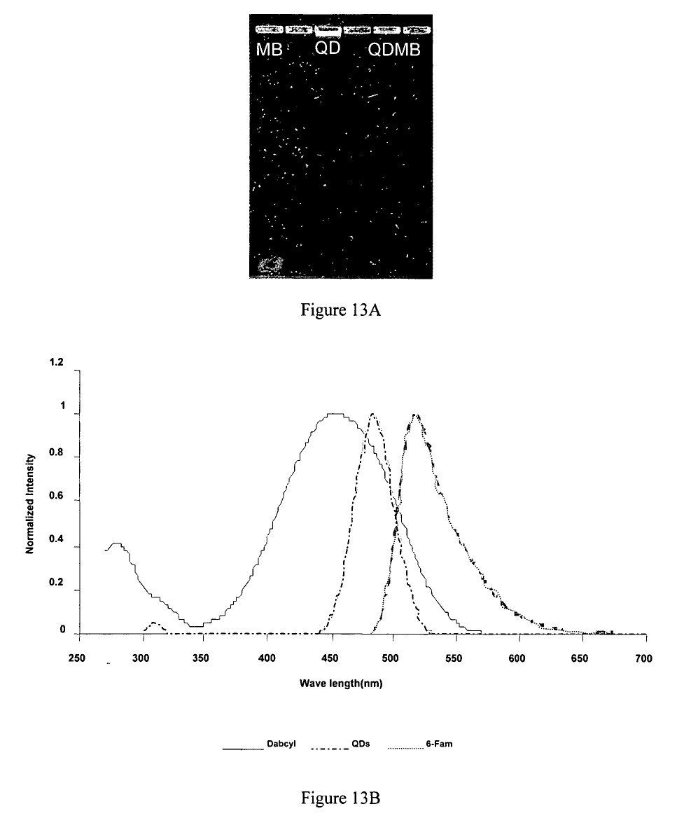

FIG. 12 summarizes this procedure. Part A of FIG. 12 shows the modification of ZnS capped CdSe QDs of about 3.7 nm size by mercaptoacetic acid. Part B of FIG. 12 shows the conjugation of QDs to MBs using EDC coupling. Part C of FIG. 12 shows the “off” state of the MB before addition of the target sequence and the “on” state after hybridization. Although FIG. 12 shows an MB wherein the first and last four bases form the stem of the MB and 17 internal bases forming a loop sequence which comprises a probe sequence, the lengths of the stem and loop may be varied as is known in the prior art. As provided herein and shown in FIG. 13A, gel electrophoresis of these hybrid MBs with the control sample showed that hybrid MBs have fairly uniform structure. Samples were run on 1% agarose gels (SeaKem Gold, Cambrex) in 10 mM K₂HPO₄ buffer (pH of 8) for 50 minutes at 100 volts. MBs without QDs and modified QDs were run as controls as well as to provide markers for their corresponding analyte molecules. Digital images were taken after staining with ethidium bromide and analyzed. The lack of additional bands suggests a lack of variations in the number of MBs attached to each QD. The single attachment of MB to each QD is also suggested by the general proximity of their corresponding bands. Furthermore, gel electrophoresis results also demonstrate covalent bonding between the QD and the MB.

One important consideration in optimal probe design is the efficiency of emission energy transfer from fluorophore, QD, to quencher. When the distance between fluorophore and quencher is in the range of about 2 to about 10 nm, the energy emitted by the fluorophore can be absorbed by the quencher if the overlap between the emission spectrum of the fluorophore and the absorption spectrum of the quencher is significant. See Kagan C. R., et al. (1996) Phys. Rev. B 54:8633; Vogelstein, B. and Kinzler, K. W. PNAS (1999) 96:9236-9241; Fang. X., et al. (2000) Anal.Chem. 72:3280-3285; and Tsuji, A., et al. (2000) Bioohys. J. 78:3260-3274, which are herein incorporated by reference. Hence, the overlap between the emission of the QD and the absorption of DABCYL was investigated and compared with 6-Fam (a commonly used organic fluorophore with close emission peak to the QD). Significant overlap (about 90%) between the emission spectra of QD and the absorption spectra of DABCYL was observed as shown in FIG. 13B. It is important to note the sharp Gaussian emission peak of the QD and the full-width-half-maximum (FWHM) of QDs and 6-Fams spectrums are about 37 nm and about 50 nm, respectively. From this one may conclude that a significant amount of emission energy can be transferred from QD to DABCYL when they are in close proximity to each other.

To predict FRET efficiency in the MB of the present invention, 3D molecular modeling was performed to visualize the relative size and topology among the stem of the MB, QD, and quencher chain. The structure of the 25 base DNA loop that forms the MB was modeled using the Biopolymers module of Insight II (Accelrys, Inc., San Diego, Calif.). Four base pairs were constrained to form a B-DNA structure for the MB stem. The remaining of the structure was subjected to several rounds of energy minimization and molecular dynamics runs to relax the MB loop structure, using the Discover module of Insight and the AMBER force field.

Molecular modeling for a macroscopic QD connected through a linear aliphatic thiol chain to the 5′ end of the MB and for a quencher chain containing DABCYL connected to the 3′ end of the MB, with their respective chemistries, was performed using the Builder and Biopolymer modules of Insight II. The QD is represented by a sphere with diameter of 3.7 nm, and was modeled as a pseudo-atom with van der Waals radius of 1.85 nm, located at an arbitrary position that accounts for the sum of the van der Waals radii of the terminal sulfur-pseudo-atom pair.

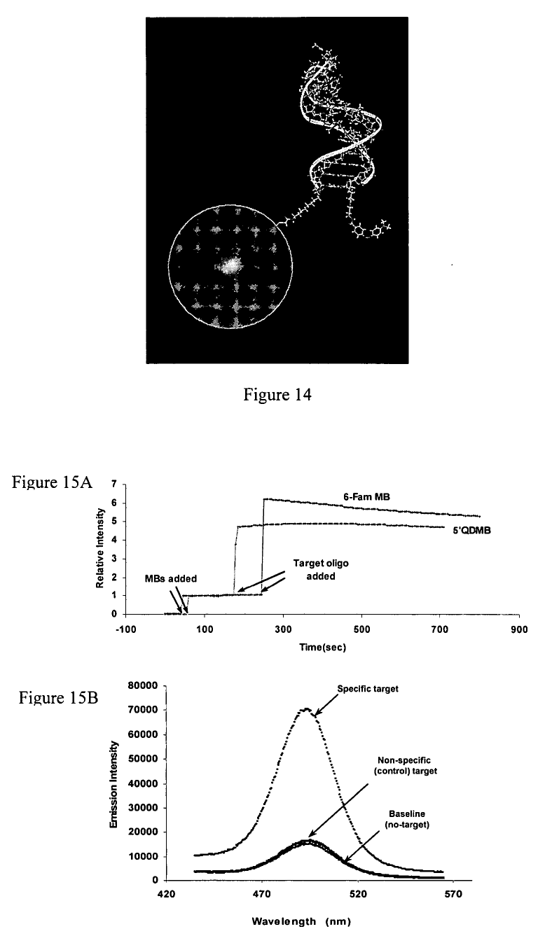

This modeling enabled the investigation of the effect of potential separation between QD and DABCYL on the emission properties of the MB. FIG. 14 shows models of the hybrid MB at two arbitrary orientations (close (FIG. 14A) and distant (FIG. 14B) separation between QD and DABCYL). The models in this figure include idealized linear representations for linkers of the QD and DABCYL with correct local geometries in the absence of non-bonded contacts. It should be noted that the MB is capable of spanning a large conformational space, mediated by the multiple rotameric states of the aliphatic thiol linker of the QD and the linker of DABCYL and by typical DNA flexibility. In essence the only space excluded from occupation is the space that would produce atomic van der Waals clashes. In these figures, the orientation of the MB depicts the formation of Watchon-Crick base pairs for the stem and a coil for the loop. A ribbon is overlaid on the DNA backbone. FIG. 14A shows QD and DABCYL in close proximity. The distance from the center of the QD to the N═N bond of DABCYL (taken as the center of DABCYL) is measured as about 3.3 nm. FIG. 14B shows this distance to be about 5.0 nm. Since the separation between the center of QD and the N═N bond of DABCYL is between about 2 to about 10 nm, it satisfies the resonance energy transfer conditions. Under these circumstances, FRET efficiency for FIG. 14A and 14B are calculated as about 54% and about 9%, respectively. The Forster radius (where transfer efficiency is 50%) is calculated as about 3.4 nm for modified QD and DABCYL in the MB. See Kagan C. R., et al. (1996) Phys. Rev. B 54:8633; Vogelstein, B. and Kinzler, K. W. PNAS (1999) 96:9236-9241; Fang. X., et al. (2000) Anal.Chem. 72:3280-3285; and Tsuji, A., et al. (2000) Bioohys. J. 78:3260-3274, which are herein incorporated by reference. During this calculation the distance from the center of QD to the N═N bond of DABCYL is used as practiced by others. See Medintz, I., et al. (2003) Nature Materials 2:630-638, which is herein incorporated by reference. The quantum yield of the modified QD is estimated as 6.8% by comparison with fluorescein whose quantum yield is 0.92 in 0.1N NaOH. See Ratilainen, T., et al. (1998) Biochemistry 37:12331-12342, which is herein incorporated by reference. Since the separation between the center of the QD and DABCYL is less than about 10 nm, the energy emitted by QD can be quenched by DABCYL via FRET. It should be noted that potential variation in FRET efficiency due to dynamic spatial organization of the probe exists. Since the torsion angles of several chemical bonds attaching to both QD and DABCYL can assume several values spanning 360°, there are many possible spatial distributions for QD modified MBs. Especially inside a buffer solution, spatial distribution varies dynamically. These two models should be considered as snapshots of a conformationally dynamic macromolecule. This could change the separation distance between QD and DABCYL. However, as modeled this distance is less than about 10 nm which allows fluorescence quenching by FRET. For simplicity, both the core (CdSe) and the cap (ZnS) of QD are shown as a perfect sphere.

For comparison, fluorescence measurements of hybrid MBs and 6-Fam attached MBs were performed. See FIG. 15A. First, a background signal from 800 μl PBS solution was measured, followed by addition of 100 μl of the MBs. The QD modified MBs were hybridized with their target sequences in a solution comprising 20 mM Tris-HCl/50 mM KCl/5 mM MgCl₂ at pH 8. The excitation wavelength for QD and 6-Fam were 350 nm and 495 nm, respectively. The emission wavelength for the QD and 6-Fam were 490 nm and 521 nm, respectively. The experiments were carried at room temperature. Concentration of target oligonucleotide and QD modified MBs were 96 nM and 10 nM, respectively. Concentration of the target oligonucleotide and the 6-Fam conjugated MB were 310 nM and 31 nM, respectively. The fluorescence from the solution before and after hybridization was recorded using Simadzu Rf-551 spectrofluorometer. The signal to noise ratio was calculated using (Fₒₙ−F_{buffer})/(F_{off}−F_{buffer}), where F means the fluorescent emission intensity. In FIG. 15, the intensity of emission is normalized. The buffer's signal was very small compared to F_{off}. Therefore, during the calculation of the signal to noise ratio, F_{buffer} was neglected.

An increase in background signal was observed for both cases. Addition of target sequence produced a significant increase in fluorescence. While the signal to noise ratio (S/N) for 6-Fam attached MB was higher than QD modified MB, QD modified MB signal remained stable over ten minutes of continuous detection. A drop in the original signal strength from about 6.5 to about 5.5 (about 15% drop in the total signal strength over ten minutes) was observed for 6-Fam attached MB. In addition, hybrid MB response time to target DNA was similar to conventional MBs. Both MBs immediately responded to their targets. This indicates similar hybridization kinetics for both MBs.

Furthermore, hybrid MBs were tested with their specific and non-specific target sequences. See FIG. 15B. The emission spectrum of 147 p mole of hybrid MBs was measured by using Fluorolog-3 (Instruments S-A. Inc. Edision, N.J.). 10 fold molar excess of the specific target and random sequence targets were used. The excitation wavelength was 350 nm.

According to Forster's theory, the efficiency E is given by

$$E = \frac{{R}_{0}^{6}}{{R}_{0}^{6} + {R}^{6}}$$

where Ro Förster radius, the distance at which transfer efficiency is 50% and R is the distance between the centers of the donor and acceptor.

Forster radius is

R₀=(8.8×10²³JK²Q₀n⁻⁴)^{1/6} Å with J in M⁻¹ cm³ units

where K² is the orientation factor for a dipole-dipole interaction , J is the spectral overlap integral, Q₀ is the quantum yield of donor without acceptor and n is the refractive index of the medium between the donor and acceptor.

Based on calculations, the overlap integral of the QD and 6-Fam are 1.32×10⁻¹³ and 6.23×10⁻¹⁴, respectively (for this calculation, K²=⅔ for random orientation, and n=1.33 were used). While the fluorescence signal increased after the addition of hybrid MBs' specific target sequence, the signal remained similar to the background signal for the non-specific target sequence test (control). This demonstrates the stability of hybrid MBs against to non-specific sequences.

In some embodiments, rather than attaching a quencher such as DABCYL at the end of stem portion of a MB, the MB may be immobilized on a gold substrate which will quench the signal from the QD when in the stem loop conformation, i.e. no bound target sequence. The distance between the gold substrate and fluorescent molecule at the end of the stem site may be optimized by changing the length of linker between the nucleic acid molecule of the MB and the gold substrate.

Alternatively, the nucleic acid molecule comprising the probe may be synthesized directly on the substrate or the probe can be synthesized separately from the substrate and then coupled to the substrate using methods known in the art. See U.S. Pat. No. 5,143,854; PCT Publ. No. WO 92/10092; Fodor et al. (1991) Science 251:767-777; PCT Publ. No. WO 90/15070; PCT Publication No. WO 93/09668; U.S. Pat. No. 5,384,261; PCT/US93/04145 and U.S. Pat. No. 5,288,514, which are herein incorporated by reference.

The MBs of the present invention may be used to assay target sequences in a test sample according to methods known in the art. The test sample comprising or suspected of comprising the target sequence may be any source of biological material which comprises nucleic acid molecules that can be obtained from a living organism directly or indirectly, including cells, tissue or fluid, and the deposits left by that organism, including viruses, mycoplasma, and fossils. The test sample can also comprise a target sequence prepared through synthetic means, in whole or in part. Typically, the test sample is obtained as or dispersed in a predominantly aqueous medium. Nonlimiting examples of the sample include blood, urine, semen, milk, sputum, mucus, a buccal swab, a vaginal swab, a rectal swab, an aspirate, a needle biopsy, a section of tissue obtained for example by surgery or autopsy, plasma, serum, spinal fluid, lymph fluid, the external secretions of the skin, respiratory, intestinal, and genitourinary tracts, tears, saliva, tumors, organs, samples of in vitro cell culture constituents including conditioned medium resulting from the growth of cells in cell culture medium, putatively virally infected cells, recombinant cells, and cell components, and a recombinant source, e.g. a library, comprising nucleic acid molecules.

The test sample can be a positive control sample that is known to comprise the target sequence or a surrogate therefor. A negative control sample can also be used which does note comprise the test sequence and is tested in order to confirm the lack of contamination by the target polynucleotide of the reagents used in a given assay, as well as to determine whether a given set of assay conditions produces false positives (a positive signal even in the absence of target polynucleotide in the sample). The sample can be diluted, dissolved, suspended, extracted or otherwise treated to solubilize and/or purify any target sequence present or to render it accessible to reagents which are used in an amplification scheme or to detection reagents. Where the sample contains cells, the cells can be lysed or permeabilized to release the polynucleotides within the cells.

The MBs of the present invention may be employed in microarrays. The MBs of the present invention with or without a quencher may be immobilized on a substrate. In some embodiments, where the quencher is not present, the substrate quenches the QD. Microarray slides attached to probe polynucleotides can be prepared using methods known in the art. See Bittner et al. (2000) Nature 406:536-540; Khan et al. (1999) Electrophoresis 20:223-9; Duggan (1999) Science 283:83-87; and DeRisi et al. (1996) Nature Genet. 14:457-60, which are herein incorporated by reference.

Kits comprising reagents useful for performing the methods of the invention are also provided. In one embodiment, a kit comprises the MBs of the present invention packaged together with instructional material. In some embodiments the kit comprises QDs, quenchers or substrates, reagents, and probes for making the MB of the present invention.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

We claim:

1. A molecular beacon comprising a quantum dot attached to a first end of at least one nucleic acid molecule having a probe and forms a stem-loop structure in the absence of a target sequence hybridized thereto and a quencher attached to a second end of the nucleic acid molecule.

2. The molecular beacon of claim 1, wherein the quantum dot is a ZnS capped CdSe quantum dot.

3. The molecular beacon of claim 1, wherein the quencher is an organic quencher.

4. The molecular beacon of claim 1, wherein the quencher is DABCYL.

5. The molecular beacon of claim 1, wherein the quencher is a gold substrate.

6. The molecular beacon of claim 1, wherein the quantum dot is a ZnS capped CdSe quantum dot and the quencher is DABCYL.

7. The molecular beacon of claim 1, wherein the quantum dot is a ZnS capped CdSe quantum dot and the quencher is a gold substrate.

8. The molecular beacon of claim 1, wherein the quantum dot has two or more nucleic acid molecules having a probe and forms a stem-loop structure in the absence of a target sequence hybridized thereto and a quencher attached to a second end of the nucleic acid molecule.

9. The molecular beacon of claim 8, wherein the nucleic acid molecules are attached to the quantum dot in a pattern.

10. The molecular beacon of claim 8, wherein the nucleic acid molecules may be the same or different.

11. The molecular beacon of claim 8, wherein the probes may be the same or different.

12. The molecular beacon of claim 8, wherein the quantum dot may be the same or different.

13. The molecular beacon of claim 8, wherein the quencher may be the same or different.

14. The molecular beacon of claim 8, wherein at least one quantum dot is a ZnS capped CdSe quantum dot.

15. The molecular beacon of claim 8, wherein at least one quencher is DABCYL or a gold substrate.

16. The molecular beacon of claim 8, wherein at least one quantum dot is a ZnS capped CdSe quantum dot and at least one quencher is DABCYL or a gold substrate.

17. The molecular beacon of claim 1, wherein the quantum dot and the quencher are operably linked, wherein in the absence of the target sequence hybridized thereto, the quencher quenches the quantum dot signal.

18. A molecular beacon array comprising a plurality of the molecular beacon of claim 1.

19. A molecular beacon array comprising a plurality of the molecular beacon of claim 8.

20. A kit comprising the molecular beacon of claim 1 and instructional material.

21. A kit comprising the molecular beacon of claim 8 and instructional material.
